(19) United States
(12) Patent Application Publication
(10) Pub. No.: US 2024/0060584 A1
(43) Pub. Date: Feb. 22, 2024

(54) SURGICAL DEVICE

(71) Applicant: GYRUS MEDICAL LIMITED, Cardiff (GB)

(72) Inventor: Christopher CHAPMAN, Cardiff (GB)

(73) Assignee: GYRUS MEDICAL LIMITED, Cardiff (GB)

(21) Appl. No.: 18/233,656

(22) Filed: Aug. 14, 2023

Related U.S. Application Data
(60) Provisional application No. 63/398,387, filed on Aug. 16, 2022.

(51) Int. Cl. F16L 27/04; A61B 18/14; B65B 55/16; F16L 33/30

(52) U.S. Cl. CPC F16L 27/04; A61B 18/14; B65B 55/16; F16L 33/30; A61B 2018/00172

(57) ABSTRACT

A connection portion for a surgical instrument provides an improved means of connection between a surgical instrument and a hose. The hose may supply irrigation fluid or may be connected to a suction source. The connection portion includes a part which is connected to and movable with the hose, ensuring that the connection between the connection portion and the hose does not limit the flow of fluid through the hose even as the hose moves. This further reduces the apparent feeling of weight caused by the hose on the surgical instrument.

### TECHNICAL FIELD

Embodiments of the present invention described herein relate to a surgical device and a connection portion for a surgical device which allows flow of fluids through a hose to the surgical device.

### BACKGROUND TO THE INVENTION

Surgical instruments, and in particular electrosurgical instruments, may include a hose or tube connection portion, allowing the supply of fluids for irrigation of a surgical site or for the removal of fluids using a suction source. This allows the surgeon operator of the instrument to clean the site of a surgical operation. However, the connection of the hose to the surgical instrument may result in the surgeon being constrained in their range of movement, the surgical tool feeling heavier, or a reduction in the flow rate through the connection region due to the movement or positioning of the hose.

There is a need for an improved connection mechanism for the connection of a surgical instrument and hose which allows improved fluid flow, improved range of movement without the snagging of the hose, and to reduce the feeling of weight as the surgical instrument moves.

### SUMMARY OF THE INVENTION

Embodiments of the present invention provide an improved connection portion for a surgical instrument or device, including a first and second part, wherein the second part can move relative to the first part. As a connected hose or tube moves, the second part of the connection portion may move and/or rotate with it, preventing snags in the hose and allowing fluid to flow easily from the hose to the surgical instrument.

A connection portion for a surgical instrument according to the present disclosure provides an improved means of connection between a surgical instrument and a hose. The hose may supply irrigation fluid or may be connected to a suction source. The connection portion includes a part which is connected to and movable with the hose, ensuring that the connection between the connection portion and the hose does not limit the flow of fluid through the hose even as the hose moves. This further reduces the apparent feeling of weight caused by the hose on the surgical instrument.

In a first aspect of the present disclosure, there is provided a connection portion for a surgical instrument, wherein the connection portion comprises: a first part, configured to allow connection of the connection portion to the proximal end of the surgical instrument; and a second part, movably connected to the first part, wherein the second part is configured to allow a hose or tube to be attached. By providing a connection portion made up of two parts which are movably connected, the second part, and the hose which may be connected to it, may spin freely, allowing the user to move the surgical instrument whilst reducing the leverage caused by the attached hose. This further prevents twisting of the hose during use, which may have caused blockages or “pinching” of the hose.

The second part may be movably connected to the first part using a ball joint. A ball joint allows the second portion to move freely. The first part may comprise a cup-shaped section and the second part may comprise a ball-shaped section, wherein the ball-shaped section fits into the cup-shaped section.

The first part may comprise a threaded section allowing connection to the surgical instrument. By providing a threaded section, the first part may be releasably connected to the surgical instrument, allowing the connection portion to be removed. This may allow improved cleaning ability, as well as allowing the connection portion and/or the surgical instrument to be replaced or repaired separately. The threaded portion ensures a good connection to the surgical instrument.

The first part may comprise a first aperture and the second part may comprise a second aperture, and wherein the first aperture and the second aperture are aligned. Providing aligned apertures allows fluids or gasses to flow through the connection portion, from an attached hose or tube to the surgical instrument.

The second aperture may be larger than the first aperture, ensuring that when the second part moves or rotates relative to the first part that the apertures are still aligned. This allows greater flow of fluids or gasses even when the second part is moved relative to the first part. If the apertures where the same size, any misalignment of the apertures would restrict the flow of fluid or gasses.

The connection portion may further comprise an O-ring, the O-ring located between the first part and the second part. The O-ring improves the seal between the first part and the second part, reducing the likelihood that a gas or liquid flowing through the connection portion escapes from the seal between the two parts.

According to a second aspect of the present disclosure, a surgical instrument may comprise the connection portion for a surgical instrument. The surgical instrument may be an electrosurgical instrument. The surgical instrument may be connected to a hose using the connection portion

According to a third aspect of the present disclosure, a method for processing an instrument for surgery comprises: obtaining the surgical instrument; reconditioning the surgical instrument; sterilizing the surgical instrument; and storing the surgical instrument in a sterile container.

### BRIEF DESCRIPTION OF THE DRAWINGS

Embodiments of the invention will now be described by way of example only and with reference to the accompanying drawings, wherein like reference numerals refer to like parts, and wherein:

FIG. 1 is a schematic diagram of an electrosurgical system including an electrosurgical instrument according to an embodiment of the present invention;

FIG. 2 is a side view of an electrosurgical instrument according to an embodiment of the present invention;

FIG. 3 is a view of the proximal end of an electrosurgical instrument including a fixed connection portion;

FIG. 4 is an alternative view of the proximal end of the electrosurgical instrument according to FIG. 3;

FIG. 5 is a view of the proximal end of an electrosurgical instrument including a movable connection portion according to an embodiment of the present invention;

FIG. 6 is a view of the proximal end of the electrosurgical instrument of FIG. 5, wherein the connection portion is shown in a different position;

FIG. 7 is a cut-away view of the proximal end of the electrosurgical instrument according to FIG. 5;

FIG. 8 is a cut-away view of a connection portion when it is not connected to an electrosurgical instrument according to an embodiment of the invention;

FIG. 9 is a view of a connection portion which is not connected to an electrosurgical instrument according to an embodiment of the invention;

FIG. 10 is a flowchart indicating a reprocessing method for the electrosurgical instrument.

### DESCRIPTION

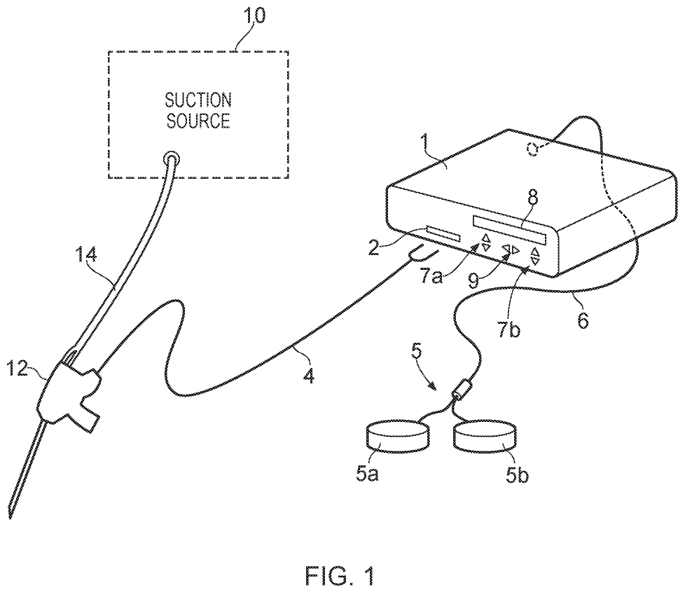

Referring to the drawings, FIG. 1 shows an electrosurgical apparatus including an electrosurgical generator 1 having an output socket 2 providing a radio frequency (RF) output, via a connection cord 4, for an electrosurgical instrument 12. The proximal end of the instrument 12 has irrigation and suction tubes or hoses 14 which are connected to an irrigation fluid and suction source 10. Activation of the generator 1 may be performed from the instrument 12 via a hand-switch (not shown) on the instrument 12, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes. Whilst an electrosurgical instrument which includes coagulation and ablation is described here, the invention is applicable to other surgical instruments which may not include coagulation or ablation, but which do require the connection of a fluid or gas-carrying hose 14 to the surgical instrument 12.

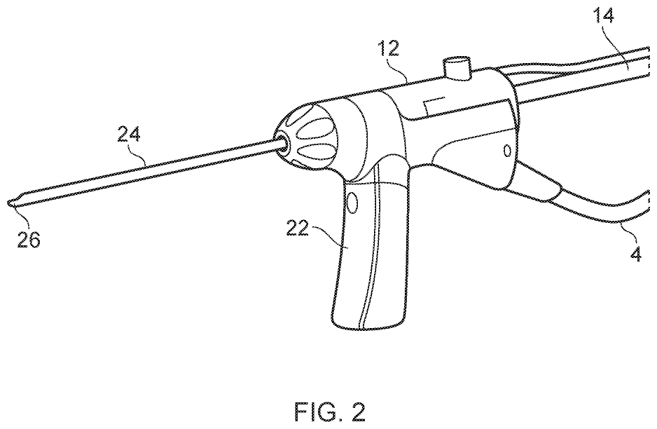

FIG. 2 shows the electrosurgical instrument 12 forming the basis of an embodiment of the present invention. The instrument 12 includes a proximal handle portion 22, a hollow shaft 24 extending in a distal direction away from the proximal handle portion, and a distal end effector assembly 26 at the distal end of the outer shaft. A power connection cord 4 connects the instrument to the RF generator 1, whereas tubes 14 (or in some cases, a single tube) connect the instrument to the irrigation and suction source 10. Alternatively, the tube 14 may be used to carry different fluids or gasses.

The connection via the tube or tubes 14 allows a surgeon to irrigate the site of surgery surrounding the distal end effector assembly 26, removing surgical debris and allowing the distal end effector assembly 26 to operate more effectively.

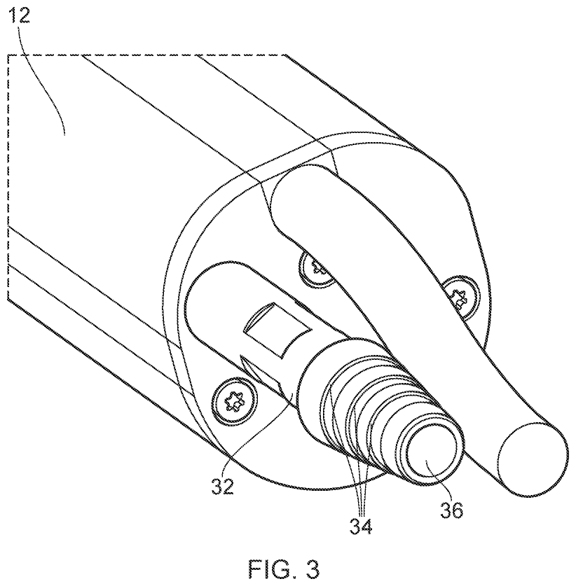

FIG. 3 shows the proximal end of the surgical instrument 12, including a connection portion 32, to which the hose or tube 14 may be attached. The connection portion 32 includes a cone-shaped proximal end, which includes a plurality of grooves 34 and an aperture 36. A hose or tube 14 may be pushed over this connection portion 32, creating a seal and allowing irrigation fluids or suction to pass through the connection portion 32, from the hose or tube 14 to the surgical instrument 12.

Typically, within prior art arrangements the connection point 32 is a fixed, non-movable, structure, to which the tubes or hose 14 are fixedly connected to the surgical instrument 12. This allows no rotation of the hose 14 when the surgical instrument 12 is rotated by the user. Further, the use of alternating grip methods can cause twisting and bending of the hose 14, which can result in the narrowing of the hose, as the hose may not be correctly centred on the aperture 36, reducing suction or delivery of irrigation fluids, and in some instances temporary blockage of the hose 14.

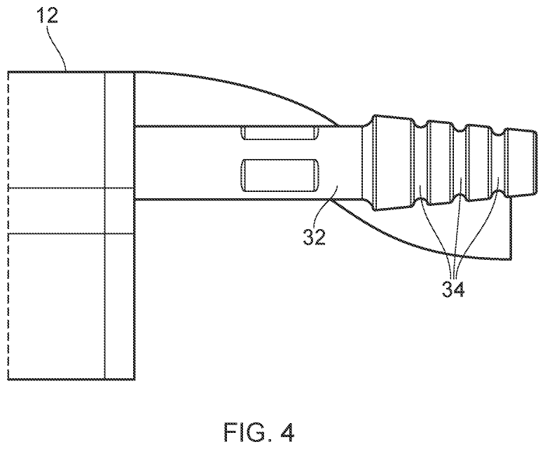

FIG. 4 shows the apparatus of FIG. 3 from a different angle. When the hose is connected to the connection portion 32, the hose 14 may be perpendicular to the proximal end of the surgical instrument 12. Due to the connection to the surgical instrument in a manner perpendicular to the proximal end of the handpiece, the surgeon may experience a “lever” issue as they move the handpiece, in which the hose 14 applies greater force to the surgical instrument as the surgical instrument is moved. This increases the apparent weight of the surgical instrument 12 and may result in the surgeon tiring faster during surgery, or the surgeon not being able to move the surgical instrument 12 in as precise a manner as required.

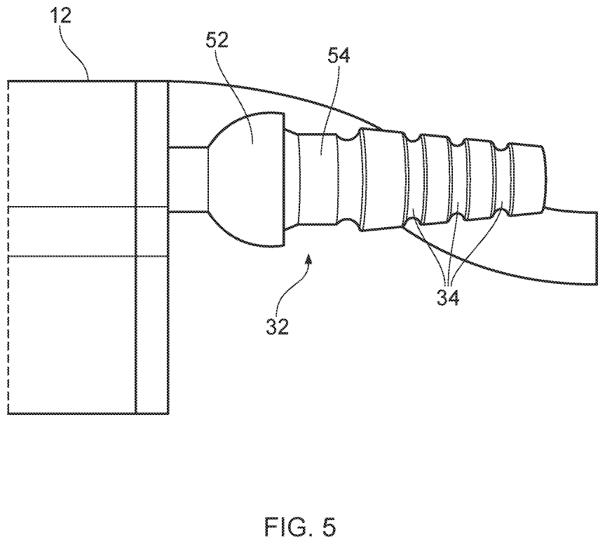

FIG. 5 shows an improved connection portion 32, constructed from two parts, a first part 52 and a second part 54 according to an embodiment of the present disclosure. The first part 52 may be fixedly or immovably attached to the proximal end of the surgical instrument 12. The second part 54 may be movably connected to the first part 52, such that the second part 54 can rotate relative to the first part 52. The second part 54 may be shaped in a conic manner, allowing a tube or hose 14 to be pushed over the conic section and the groves 34 of the second part 54 until a seal is made.

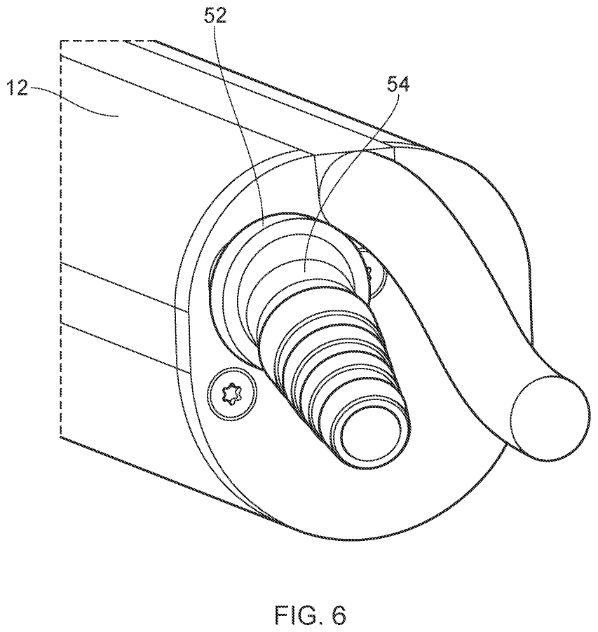

FIG. 6 shows a different view of the proximal end of the surgical instrument 12 shown in FIG. 5. The second part 54 of the connection portion is shown in a different position to that of FIG. 5, such that the second part 54 has moved relative to the first part 52 of the connection portion whilst still remaining attached to the first part 52.

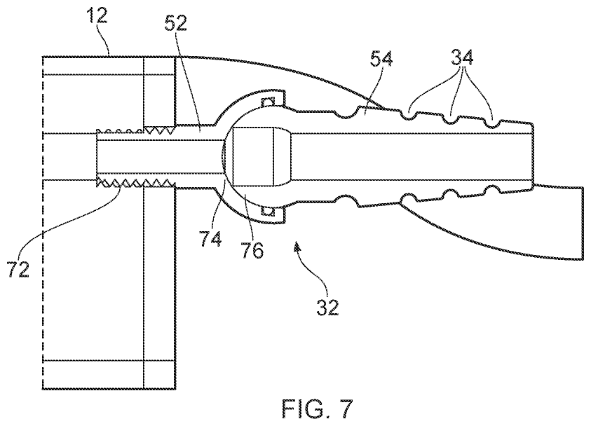

FIG. 7 shows a cut-away view of the apparatus of FIG. 5. The first part 52 of the connection portion may include a threaded section 72. The threaded section 72 may allow the first part 52 to be fixedly connected to a corresponding threaded section in an aperture on the proximal end of the surgical instrument 12. This ensures that the first section is connected in a manner which allows simple construction whilst also ensuring that the first part 52 is immovably connected during use.

To allow movable connection of the first part 52 and the second part 54 of the connection portion, the connection portion may include a ball-joint. The first part 52 may include a cup-shaped section 74 and the second part 54 may include a ball-shaped section 76. The ball-shaped section is shaped in such a way that it corresponds to the shape of the cup-shaped section 74, such that the ball-shaped section 76 may fit into, or connect with, the cup-shaped section 74.

The connection between the first part 52 and the second part 54 using a cup-shaped section 74 and a ball-shaped section 76 allows the second part to move as a hose or tube 14 which is connected to it moves, in that the ball-shaped section 76 can rotate within the cup-shaped section 74. The ball joint allows the second part 54 to spin freely through the ‘X’ axis, preventing twisting in the hose 14 which is connected to the second part. For example, the ball joint allows the second part 54 up to 40° of movement relative to the first part 52. As the second part 54 is connected to a hose or tube 14, as the user moves the surgical instrument, the hose may rotate or move with the second part 54 whilst retaining a seal on the second part 54. The angle change of the second part 54 reduces the lever issue and makes the device seem lighter without removing any material.

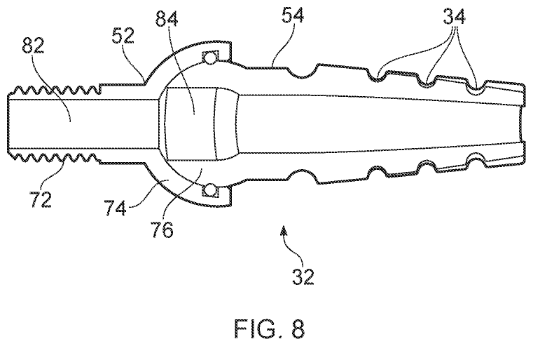

FIG. 8 shows a view of the connection portion 32 when it is not attached to a surgical instrument 12. The first part 52 includes an aperture 82 and the second part 54 includes an aperture 84. When the first part 52 and the second part 54 are connected, the first aperture 82 and the second aperture 84 are aligned, such that irrigation fluid or air may flow through, and between, the first part 52 and the second part 54. This allows fluid or suction to be applied from a suction source to the surgical instrument through the connection portion.

The second aperture 84 may be larger than the first aperture 82. This ensures that even when the second part 54 has moved, due to pressure from the connected hose 14, that the first aperture 82 and the second aperture 84 remain aligned in such a way that the first aperture 82 is always aligned with the second aperture 84. This prevents a situation in which the movement of the hose results in a pinching-off of flow, such that the flow which may pass through the connection portion 32 is always, and only, limited by the size of the aperture in the first part 52 of the connection portion 32.

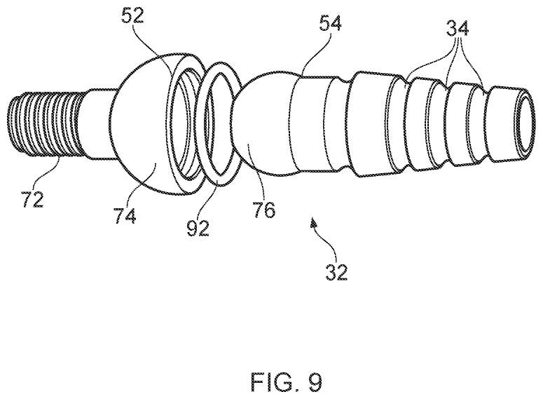

FIG. 9 shows a deconstructed view of the connection portion 32. In some instances, the connection portion may further include an O-ring 92, as well as the first part 52 and the second part 54. The O-ring 92 is provided between the first part and the second part, providing an improved seal compared to examples where the O-ring is not present. This ensures that the second part 54 can move as the connected hose or tube 14 moves, whilst still ensuring that the seal provided by the connection portion 32 allows no or minimal leakage of irrigation fluid or air.

Whilst the description presented herein refers primarily to an electrosurgical instrument, the description is also applicable to surgical instruments which are not electrosurgical.

The electrosurgical instrument devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

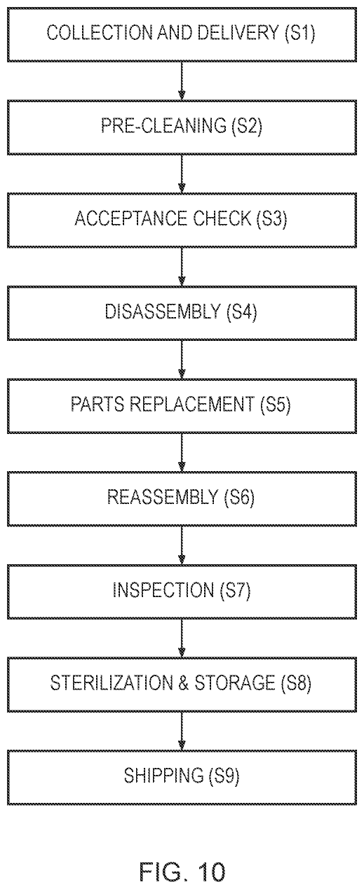

In more detail, the electrosurgical instrument 12 described above may be disposed of after one use, or may be repeatedly used a plurality of times. In the case of a configuration that is repeatedly used a plurality of times, for example, reprocessing method shown in FIG. 10 is required. An operator collects the used treatment instrument 12 after it has been used for treatment and transports it to a factory or the like (Step S1). Then, the operator cleans and sterilizes the collected and transported used treatment instrument 12 (Step S2). Next, the operator performs an acceptance check of the used treatment instrument 12 (Step S3). Subsequently, the operator disassembles the used treatment instrument 12 (Step S4) and replaces some parts of the used treatment instrument 12 with new parts (Step S5). After step S5, the operator assembles a new treatment instrument 12 (Step S6). In some examples, Step S6 can include adding an identifier to indicate the device has been modified from its original condition, such as a adding a label or other marking to designate the device as reprocessed, refurbished or remanufactured. After Step S6, the operator sequentially performs an inspection (Step S7), sterilization and storage (Step S8), and shipping (Step S9) of the new treatment instrument 12.

Preferably, the surgical instrument described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

Various modifications whether by way of addition, deletion, or substitution of features may be made to above described embodiment to provide further embodiments, any and all of which are intended to be encompassed by the appended claims.

1. A connection portion for a surgical instrument, wherein the connection portion comprises:
a first part, configured to allow connection of the connection portion to the proximal end of the surgical instrument; and
a second part, movably connected to the first part, wherein the second part is configured to allow a hose or tube to be attached.

2. The connection portion according to claim 1, wherein the second part is movably connected to the first part using a ball joint.

3. The connection portion according to claim 2, wherein the first part comprises a cup-shaped section and the second part comprises a ball-shaped section, wherein the ball-shaped section fits into the cup-shaped section.

4. The connection portion according to claim 1, wherein the first part comprises a threaded section allowing connection to the surgical instrument.

5. The connection portion according to claim 1, wherein the first part comprises a first aperture and the second part comprises a second aperture, and wherein the first aperture and the second aperture are aligned.

6. The connection portion according to claim 5, wherein the second aperture is larger than the first aperture.

7. The connection portion according to claim 1, wherein the connection portion further comprises an O-ring, the O-ring located between the first part and the second part.

8. A surgical instrument, the surgical instrument comprising the connection portion according to claim 1.

9. The surgical instrument according to claim 8, wherein the surgical instrument is an electrosurgical instrument.

10. The surgical instrument according to claim 8, wherein the surgical instrument is connected to a hose using the connection portion.

11. A method for processing an instrument for surgery, the method comprising:
obtaining the surgical instrument of claim 1;
reconditioning the surgical instrument;
sterilizing the surgical instrument; and
storing the surgical instrument in a sterile container.
